(19) United States
(12) Patent Application Publication
(10) Pub. No.: US 2010/0068691 A1
(43) Pub. Date: Mar. 18, 2010

(54) TISSUE SYSTEM AND METHODS OF USE

(75) Inventors: Hanry Yu, Singapore (SG); Yuet Mei Khong, Singapore (SG)

Correspondence Address: INTELLECTUAL PROPERTY / TECHNOLOGY LAW, PO BOX 14329, RESEARCH TRIANGLE PARK, NC, 27709, US

(73) Assignee: AGENCY FOR SCIENCE, TECHNOLOGY AND RESEARCH, Centros (SG)

(21) Appl. No.: 11/577,091

(22) PCT Filed: Oct. 7, 2005
(86) PCT No.: PCT/SG05/00346

§ 371 Date: Jul. 2, 2007

Related U.S. Application Data
(60) Provisional application No. 60/618,030, filed on Oct. 12, 2004.

(51) Int. Cl. A01N 1/02; C12M 3/00

(52) U.S. Cl. 435/1.2; 435/284.1

(57) ABSTRACT

Apparatus and methods of use of a tissue system for culture and perfusion. The apparatus comprises needles for injecting a fluid into the tissue.

### FIELD OF THE INVENTION

This invention relates generally to a tissue system.

### BACKGROUND OF THE INVENTION

Otto Warburg initiated the first attempt to culture organoid slices in vitro using liver slices (Warburg, O., (1923), Biochemische Zeuschrift 142: 317-333). Follow-up research had solved some existing problems in that area such as irreproducible tissue thickness and mechanical damage to tissues using the Krumdieck slicer (Krumdieck et al, (1980), Analytical Biochemistry 104: 118-123). However, with the advent of established cell isolation techniques, modem research has relied on cell cultures, such as hepatocyte cultures, as a platform for experimental investigations. However, the cell isolation process not only damages cells, such as the plasma membrane, but it can also irreversibly disrupt the cell polarity and dynamics as a result of the destruction of anchorage points provided by innate extracellular matrices.

### SUMMARY OF T HE INVENTION

Accordingly, the inventors have succeeded in devising a novel tissue culture and/or perfusion technique that exploits the inherent tissue matrix and angio-architecture of tissue slices and concurrently, enables, for example, long-term maintainance of viable, functional cells. This technique utilizes micro-fabricated needles as a perfusion platform to interface with the existing micro-vasculature of tissue slices. For example, liver slices and micro-needles can be embedded in between a PDMS membrane and glass cover slip to sustain adequate pressure within the tissue slice. Utilization of tissue slices provides, for example, the advantage of cellular heterogeneity and interactions within an intact cellular matrix. Integration of micro-needles can, for example, serve as a substitute for the larger preceding vasculatures that supplements nutrients to the cells. Also, for example, the flow rate and/or pressure of the inlet fluids and nutrients can be controlled or adjusted to allow uniform distribution of fluids and nutrients to the tissue sample via inherent pathways. Such control can also, for example, serve to reinstate the inherent hemodynamic environment of the tissue. For example, in the case of liver tissues, by controlling the flow rate and pressure of the inlet fluids and nutrients, the present system not only can allow for uniform distribution of nutrients to the entire construct via inherent sinusoidal pathways, but also the reinstatement of the inherent hemodynamic environment of the liver.

### BRIEF DESCRIPTION OF THE DRAWINGS

The skilled artisan will understand that the drawings, described below, are for illustration purposes only. The drawings are not intended to limit the scope of the present teachings in any way.

FIG. 1 shows an exemplary system of the invention that is used in Example 1.

FIG. 2 is an illustration of a 2 mm thick tissue slice perfused with 10% Trypan Blue for 5 minutes using a single 30 G needle. (a) Top of the tissue, (b) bottom of the tissue and (c) cross-sections of the tissue at the needle puncture point.

FIG. 3 is an illustration of a 2 mm thick tissue slice perfused with 10% Trypan Blue for 5 minutes using a single 30 G needle. (a) Bottom of the tissue (completely perfused), (b) Top of the tissue, (c) schematic of cross section, (d) Cross section A (L), (e) Cross section A (M), (f) Cross section B (M) and (g-h) Cross section B (R).

FIG. 4 is an illustration of incubation systems; (a) stationary system, (b) rocker dynamic organ culture.

FIG. 5 is an illustration of the effect of different incubation systems (stationary system and rocker system) using MTT assay. Slices were perfused with UW solution, precision-cut to 300 μm using Krumdieck slicer and pre-incubated for 1 hr.

FIG. 6 is an illustration of a 300 um thick tissue stained with 100 uM Rho 6G and captured using confocal microscopy (excitation: 543 nm and emission: 560 nm). Each stack is 150 μm with optical sections captured every 2 μm. (a) and (c) were stained under rocking conditions and (b) and (d) were stained under static diffusion condition. (a-b) Images obtained from the bottom layer of the tissue and (c-d) Images obtained from the top layer of the tissue.

FIG. 7 is an illustration of a 2 mm thick tissue stained with 100 uM Rho 6G and captured using confocal microscopy (excitation: 543 nm and emission: 560 nm). Each stack is 150 μm with optical sections captured every 2 μm. (a) and (c) were stained using single needle perfusion and (b) and (d) were stained under rocking conditions. (a-b) Images obtained from the bottom layer of the tissue and (c-d) Images obtained from the top layer of the tissue.

FIG. 8 is an illustration of a) micro-needle chamber components; b) Part 1—Base; c) Part 2—PDMS membrane; d) Part 3—micro-needle platform; e) Part 4—Top cover.

FIG. 9 is a schematic representation and assembly diagram of the micro-needle apparatus.

FIG. 10 is micro-needle apparatus set-up.

FIG. 11 is an illustration of 900 μm liver slices perfused with 10% Trypan Blue; (a) Top of the liver slice; (b) Bottom of the liver slice, showing the cutting line of the cross section; (c) Left side of the liver slice (L); (d) Right side of the liver slice (R). Small arrows show regions that remain to be perfused.

FIG. 12 is an illustration of 900 μm liver slices perfused with 10% Trypan Blue; (a) Top of the liver slice; (b) Bottom of the liver slice, showing the cutting line of the cross section; (c) Left side of the liver slice (L); (d) Right side of the liver slice (R). Small arrows show regions that remain to be perfused.

### DETAILED DESCRIPTION OF THE INVENTION

Thus, in certain aspects, the present invention offers a solution to the mass transfer limitation conundrum that had plagued the field of tissue slice engineering for many years. In some aspects, the present invention provides a higher level of biomimicry by exploiting existing inherent extracellular matrix and microvasculature of a tissue such as, for example, the liver. In some aspects, the present invention excludes the necessity of cell isolation and stimulation of cells to maintain high functionality with a variety of growth factors, scaffold design, and co-culture. Micro-needle perfusion enhances the uniform distribution of perfusion media, which subsequently ameliorates the viability and functionality of the tissue over a long-term culture. (Example 1 and 2). Micro-fabrication techniques enable design and development of a range of micro-needles with varying size, array distance and shape, which permits the versatility of experimental designs. Utilization of micro-needles can potentially facilitate the introduction of different drugs at different regions of the liver, and investigate the interactions of the cells from different regions with respect to the drugs introduced.

The current invention marks the inauguration of a living tissue biochip with the advantages of a compact, high throughput platform and with at least the following applications, for example:

### Platform for ADME/tox Investigations and High Throughput Screening (HTS)

ADME/tox is concerned with how various factors, such as a drug, for example, are adsorbed, distributed, metabolized, and/or eliminated and any harmful or toxic properties of a factor and its metabolites. For example, the application of liver slices in ADME/tox studies can be an experimental tool. However, culture of liver slices over a period of time can be unfeasible due to necrotic tissues in the central region of the tissue slice as a result of mass transfer limitations. The current invention can provide a solution to this problem and hence, creates a new paradigm to ADME/tox experimental designs.

Historically, due to the short life-time of tissue slices, drug toxicity tests were conducted in non-physiologically high dosage. Such tests only offer a very superficial understanding of the actual drug metabolism. The introduction of a long-term tissue biochip enables experimental designs that utilize more realistic and physiological dosage and thus, allows more in-depth studies to be performed.

The micro-needles system can also be used to inject different pharmaceutical biomolecules into different parts of the tissue, creating a differential concentration and type of drugs within the same tissue slice. This technique can assist the understanding of interaction between different kinds of chemicals and how these chemicals affect living tissue by being differentially distributed in different parts of the slice.

The technology of micro-fabrication also offers the possibility of integrating in situ and real-time sensors, which can detect hormones, oxygen levels, ligands and chemical agents.

Chip-based systems can be easily duplicated and multiplexed, facilitating the integration of HTS to screen potential pharmaceutical products. Such systems offer the advantage of speed, flexibility and accuracy in evaluating the pharmacokinetics of a particular drug.

### Tissue Biochip for Bioimaging and Biological Investigations

Utilization of a thick tissue that is embedded in between a transparent PDMS membrane and cover slip permits the incorporation of confocal microscopy and multiphoton microscopy as a bioimaging tool. Integration of these experimental techniques along with the current chip-based tissue enables at least the following applications, for example:

(i) By using micro-needles to interface with the existing angio-architecture of the liver slice, it is possible to observe the metabolism of a drug in an in vivo environment to the extent of a single cell resolution. The entire biotransformation and transport pathway of a single or multiple fluorescent-tagged biomolecule can be tracked and imaged online and in real-time.

(ii) Since a tissue slice retains the complex tissue matrix and cell heterogeneity, the interactions between different cell types can be observed. In addition, the interactions and in vivo dynamic cellular changes in the introduction of a foreign substance such as drugs or metastatic cancer cells can be observed.

(iii) Since micro-needles offer the advantage of differential introduction of multiple drugs, this chip-based device can be used to observe not only the effect of the drugs at a specific region, but also the interaction of cells with the drug and among different cell types at the interface region.

### Tissue Microarrays

This chip-based device can be multiplexed to form tissue microarrays (TMA). TMA is normally used for high throughput histological studies, however, existing TMA utilizes thin sections of fixed tissues. The current device can also be used for similar applications with the advantage of thicker tissue sections and also viable, functional tissues. This advantage presents many applications such as, for example:

(i) Viable tissue sections can be cryopreserved and commercially marketed. Viable and functional tissues preserved this way enable off-the-shelf availability of tissue chips for experiments, avoiding the need for cell or tissue isolations. This not only permits histological studies, but also functional studies.

(ii) Thin tissue sections were traditionally preferred due to inability to uniformly stain the entire tissue. With the current invention using micro-needles perfusion, this problem can be eliminated.

### Cell Culture Analogues

Besides using the liver as a sample source, the current technique can also be extended to other organs of the body such as the lung and the kidneys. In the past, cell culture analogues (CCA) of the body had been created using in vitro cell culture flasks containing different parenchymal cells obtained from vital parts of the body. A chip-based CCA has also been introduced recently with the benefit of physiologically representative flowrates and shear forces [Sin et al; (2004); Biotechnology Progress; 20; pp. 338-345]. A similar CCA can be created using the current technique, i.e. isolating representative tissue slices from vital parts of the body such as the liver, lungs and kidneys and interfacing these tissues slices via micro-needles. The advantage of this tissue chip-based CCA relative to previous designs is the utilization of a highly biomimicry cellular construct comprising both parenchymal and non-parenchymal cells.

### Engineering Large Tissue Constructs

By adjusting the densities and the length of micro-needles, we can culture tissue slices, and engineered tissue constructs of much larger dimensions (thicker and bigger) than currently possible. In the current culture configurations (either static or dynamic), tissues or tissue constructs larger than 1 mm typically disintegrate rapidly due to limited mass transfer through these pieces of tissue constructs. Perfusion through micro-needles can be precisely controlled to provide nutrients and remove metabolic wastes for efficient functions of cells and maintenance of structural integrity of tissues or tissue constructs of large dimensions >1 mm.

Silicon microfabricated micro-needles and PDMS chamber can be replaced with biodegradable polymers. Utilization of a porous biodegradable material can enable the live cells of the tissue to grow into and occupy the porous structure, hence, making it possible to grow a small tissue slice into a larger tissue slab. The biodegradable material can be seeded with stem cells or progenitor cells prior to encapsulating the tissue slice. In this configuration, the stem cells or progenitor cells can provide a cell source for proliferation, and the liver slice can provide signals for the cells to differentiate. By using the abovementioned methods to grow a larger tissue slab, it can be used for bioartificial liver and other tissue engineering applications to substitute damaged organ parts.

The headings (such as “Background of the Invention” and “Summary of the Invention”) used herein are intended only for general organization of topics within the disclosure of the invention and are not intended to limit the disclosure of the invention or any aspect thereof. In particular, subject matter disclosed in the “Background of the Invention” may include aspects of technology within the scope of the invention and may not constitute a recitation of prior art. Subject matter disclosed in the “Summary of the Invention” is not an exhaustive or complete disclosure of the entire scope of the invention or any embodiments thereof.

The citation of references herein does not constitute an admission that those references are prior art or have any relevance to the patentability of the invention disclosed herein. All references cited in the specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations of the stated features. Specific Examples are provided for illustrative purposes of how to make, use and practice the compositions and methods of this invention and, unless explicitly stated otherwise, are not intended to be a representation that given embodiments of this invention have, or have not, been made or tested.

### EXAMPLES

The following examples are intended to be illustrative and are not intended to limit the scope of the invention.

### Example 1

Perfusion studies using Trypan Blue.

Long-term tissue culture of thick tissue slices has always been the holy grail of tissue slice tissue engineering. Thick tissue slices has been shown to possess better morphology and functionality (Shigematsu et al, Experimental and Molecular Pathology 69: 119-143 (2000)), however, the culture duration is limited due to mass transfer limitations. Utilization of dynamic cultures enhances mass transfer, but exposes the tissue to mechanical abrasions and damage. Embedding tissue slices in agarose has been shown to protect the tissue and hence, improve viability and functionality to the extent of prolonging the survival of the tissue (Nonaka et al, Cell Transplantation 12: 491-498 (2003)). The current example illustrates how a single micron-sized needle can be used to perfuse a thick liver slice under static and embedded conditions.

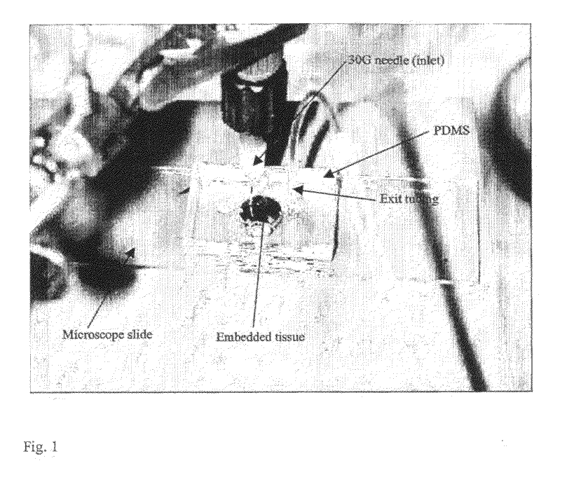

Livers perfused with 4% formalin at 37° C. were excavated from Male Wistar rats (weight of approximately 250 g) that were anaesthetized using sodium phenobarbitone and injected with 0.5 mL heparin. Tissue cylinders from liver samples were prepared using an 8-mm diameter coring tool on a motor-driven tissue coring device. Tissue slices were precision-cut to 2 mm using a vibratome (DTK-1000, Pelco International, Redding, USA). 10% Trypan Blue dye was perfused into the 2 mm thick tissue slice using a set-up as shown in FIG. 1.

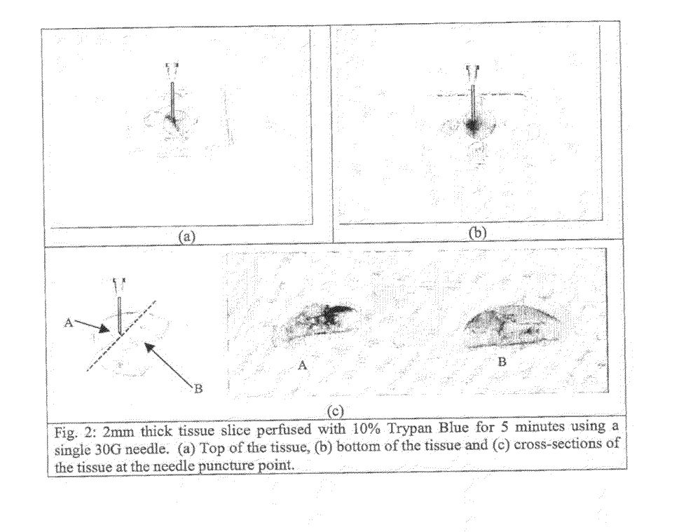

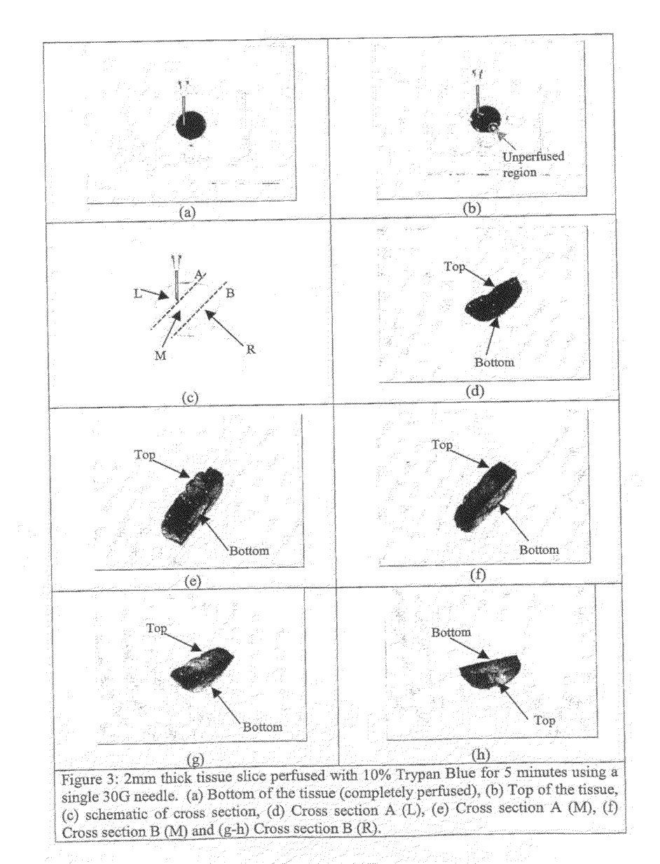

FIG. 2 shows the 2 mm thick tissue slice that has been perfused with Trypan Blue for 5 minutes. As shown in FIG. 2(c) the dye has penetrated the entire cross section of the tissue particularly at the needle puncture point. Using a similar configuration, when the perfusion was conducted for 1 hour, the entire tissue was stained with Trypan Blue (FIG. 3). Since using a single needle exhibit enhanced mass transport efficiency, utilization of an array of micro-needles can achieve higher efficiency and eliminate the unperfused regions.

### Example 2

Perfusion studies using Rho 6G and correlation to liver slice viability. Enhanced mass transfer of nutrients and removal of wastes is often correlated to improved viability and functionality of living cells and tissues. Example 1 illustrates how a single micro-needle can be used to interface with existing microvasculature and hence, perfuse through the sinusoidal pathways. The current example aims to illustrate the correlation between improved perfusion and mass transfer to the viability of liver slice.

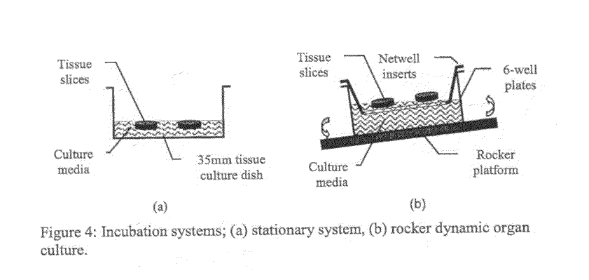

Livers perfused with UW solution at 4° C. were excavated from Male Wistar rats (weight of approximately 250 g) that were anaesthetized using sodium phenobarbitone and injected with 0.5 mL heparin. Tissue cylinders from liver samples were prepared using an 8-mm diameter coring tool on a motor-driven tissue coring device. Tissue slices were precision-cut to 300 μtm using Krumdieck slicer (Alabama Research and Development, Germany). Slices were cultured under static conditions and rocking conditions (Leeman et al, 1995, Toxicity in Vitro 9; pp. 291-298) as illustrated in FIG. 4; in Hepatozym-SFM (Gibco Laboratories) supplemented with 100 U/mL penicillin, 100 u g/mL streptomycin, 0.1 uM dexamethasone, 20 ng/mL EGF and 10 uM insulin. Both systems were incubated at 37° C. and 95% O₂, 5% CO₂. Tissues were removed from incubation at an interval of −1 hr (before pre-incubation), 0 hr (after pre-incubation), 3 hrs, 6 hrs and 24 hrs and assayed using MTT assay. Fixed liver samples were obtained using similar method as described in Example Tissue slices were precision-cut to 300 μm using Krumdieck slicer (Alabama Research and Development, Germany) (Krumdieck et al, 1980) or 2 mm using a vibratome (DTK-1000, Pelco International, Redding, USA). 2 ml of 100 μM Rho 6G dye was perfused into the 2 mm thick tissue slice using the set-up as shown in FIG. 1. Diffusional studies were performed by incubating 300 μm and 2 mm slices in 3 ml of 100 μM Rho 6G under static and rocking conditions. Stained slices were imaged under confocal microscopy (Zeiss LSM 510) using 10× objective at excitation wavelengths and emissions wavelengths of 543 nm and 565 nm respectively. For each tissue slice, a stack of 76 optical sections was captured at every 2 μm increment (total thickness of each stack is 150 μm).

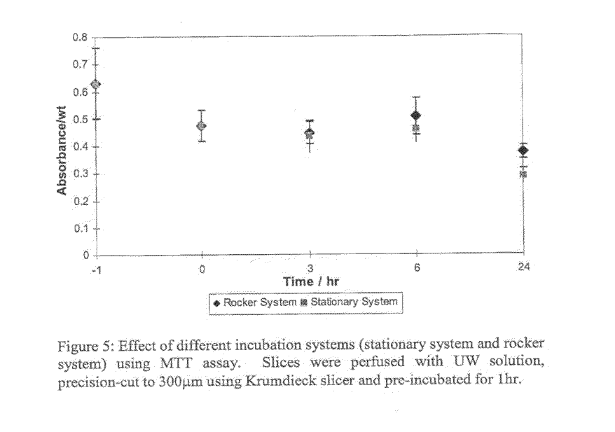

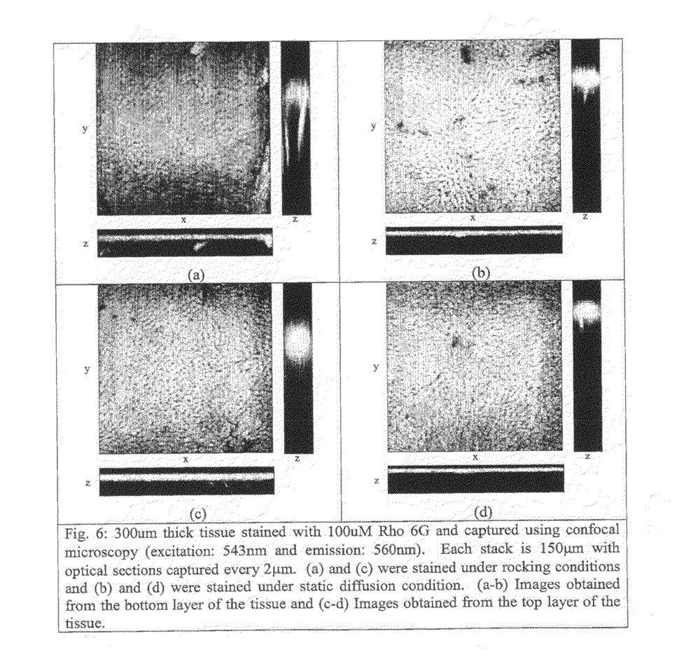

In FIG. 5, it can be observed that incubation under rocking conditions improved the survival of the tissue, particularly after a 24 hr culture. The improved viability can be correlated to the diffusion of the nutrients into the tissue, as illustrated by analogous diffusion investigations using Rho 6G in FIG. 6. This figure demonstrates the penetration of the dye after 1 hour incubation into the tissue for both static and rocking conditions. Under static conditions, the dye seems to accumulate at a short distance from the surface of the tissue, hence giving a thin highly fluorescent layer. In comparison to the rocker system, the dye penetration is more diffused, resulting in a lower intensity but thicker fluorescent layer.

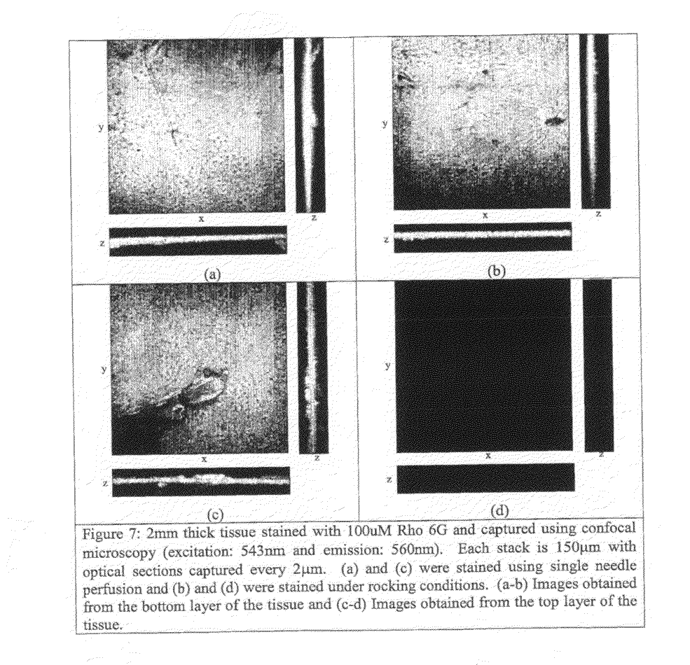

Using this correlation, a similar diffusional investigation using a 2 mm thick liver slice and under needle perfusion was conducted (as illustrated in FIG. 7). Results indicate that using a micro-needle perfusion enables the dye to penetrate to at least a depth comparable to the rocker system. This is illustrated by the thick diffuse fluorescent layer on both the top and bottom layer of the tissue. The needle perfusion also possesses a significant advantage to perfuse thick tissue sections in comparison to the rocker system. As shown in FIGS. 7(c) and (d), the top layer of the needle perfused tissue is uniformly stained, however, the rocker incubated tissue is very faintly stained.

The above studies establish that a single micron-sized needle can be used to interface with the existing microvasculature of the tissue slice and thus, enable efficient perfusion for nutrients delivery and waste removal. This efficiency can be further enhanced with the integration of an array of micro-needles. Moreover, micro-needle perfusion provides a platform to eliminate mass transfer limitations for thick tissue sections, consequently, improving the survival of thick tissue sections over a long-term culture.

Embedding tissues in a PDMS chamber can be an option that can, for example, protect the surface of the tissues from mechanical abrasion and damage, hence, reducing apoptotic signals from the surface that can result in degenerative tissues.

### Example 3

Perfusion using micro-needle chamber.

Examples 1 and examples 2 show perfusion of a liver slice using a single micro-needle. The current example illustrates the perfusion of the liver slice using a fabricated micro-needle chamber.

### Methodology

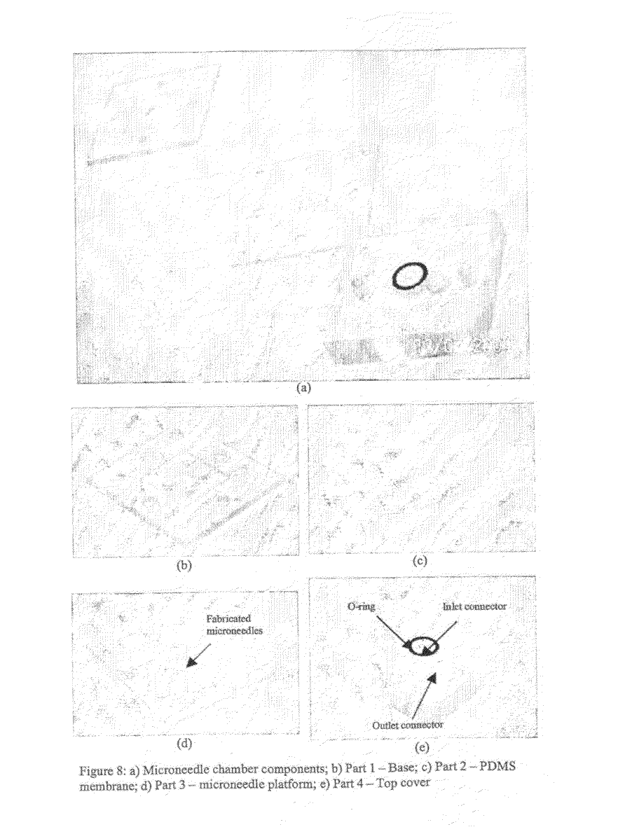

Fabrication of micro-needle chamber. The micro-needle chamber comprises of 4 parts (FIG. 8):
Part 1—base: This part is designed to hold a 22 m×22 mm coverslip and the PDMS membrane.
Part 2—PDMS membrane: This membrane is designed to hold the 8 mm tissue slice.
Part 3—Micro-needle platform: A micro-needle array comprising of 4 needles is CNC fabricated into this platform.
Part 4—Top cover: The top cover is designed to enclose the chamber and for connections to the perfusion circuit.

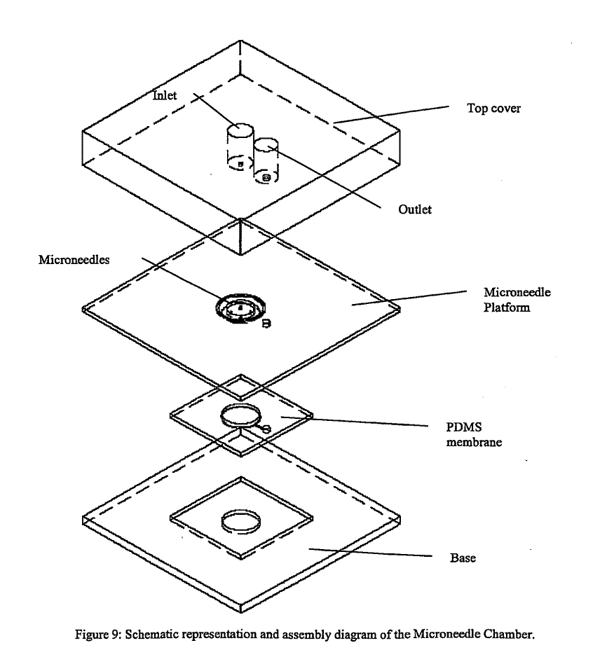

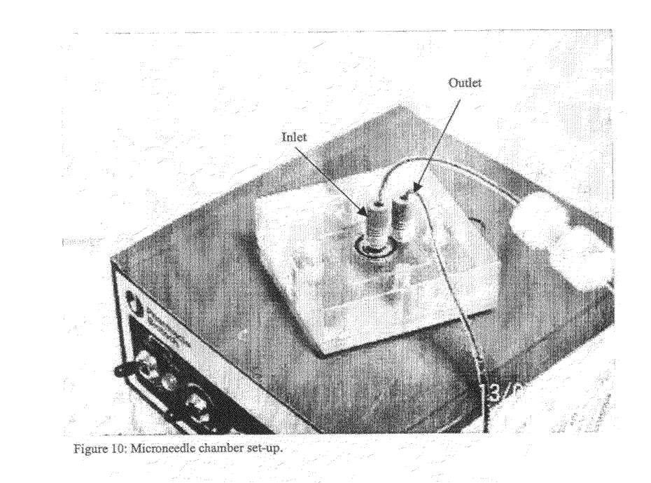

A schematic representation of the micro-needle chamber and its assembly is as shown in FIG. 9. The chamber is fixed together using M4 screws and connected to the perfusion circuit as shown in FIG. 10. Fluid is pumped from a reservoir by a peristaltic pump (P-1, Amersham) to enter the chamber via a center inlet, exits via a side outlet and is returned to the reservoir.

Diffusional Studies

Liver slices are prepared by excavating UW solution perfused liver from Wistar rats (250-300 g) and sliced to 900 μm using the Krumdieck slicer (Alabama Research and Development, Germany). 3 ml of 100 μM Rhodamine 6G dye or 10% Trypan Blue was perfused into the 900 μm thick tissue slice for 1 hour using the set-up as described above. A static control was set up by incubated a 900 μm tissue slice in 3 ml of Rhodamine 6G. Rhodamine 6G stained slices were imaged under confocal microscopy (Zeiss LSM 510) using 10× objective at excitation wavelengths and emissions wavelengths of 543 nm and 565 nm respectively. For each tissue slice, a stack of 76 optical sections was captured at every 2 μm increment (total thickness of each stack is 150 μm).

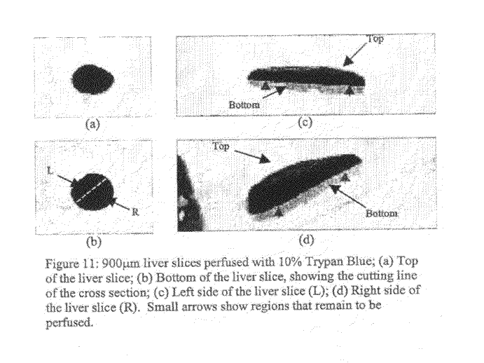

FIG. 11 shows the diffusional results of a 900 μm liver slice perfused with 10% Trypan Blue using the micro-needle chamber. As shown in FIG. 11a, the top of the tissue is entirely perfused, whereas the bottom of the tissue (FIG. 11b) is partially perfused. Cross sections of the tissue (as shown in FIGS. 11c and d) show that the dye has penetrated into deeper layers of the tissue (note: several regions remain to be perfused as indicated by the small arrows in the figure).

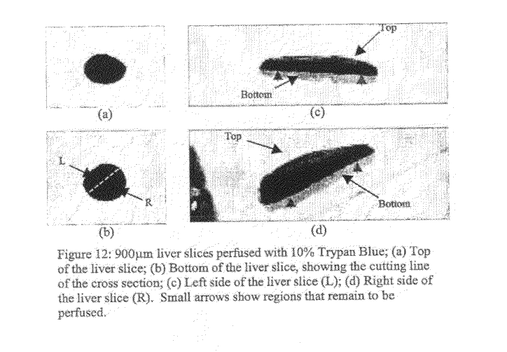

Rhodamine 6G diffusional studies of the perfusion system and the static culture results are shown in FIG. 12. Diffusion into the tissue slice using the micro-needle perfusion system demonstrates that the dye has penetrated into the liver slice. In comparison to the static culture, dye penetration in the micro-needle perfusion system is observed to be more diffused and penetrated deeper than the static system.

This example shows the possibility of utilizing a micro-needle array fabricated into a micro-needle chamber to perfuse a thick tissue slice. Diffusional studies show that the penetration of dye is improved in comparison to a static system, thus, demonstrating improvement in mass transfer.

All references cited in this specification are hereby incorporated by reference in their entirety. The discussion of the references herein is intended merely to summarize the assertions made by their authors and no admission is made that any reference constitutes prior art relevant to patentability. Applicant reserves the right to challenge the accuracy and pertinence of the cited references.

1. A tissue system comprising:
a chamber for containing the tissue;
an outlet port fluidly coupled to the chamber;
an inlet port fluidly coupled to the chamber; and
one or more micro-needles each comprising a tip end, the tip end being positioned about the inlet port and configured for injecting a fluid into a portion of the tissue.

2. The system of claim 1 wherein the inlet port, the chamber, and the outlet port are configured to provide a continuous flow of the fluid through the tissue.

3. The system of claim 2 wherein the continuous flow has a flow rate substantially equivalent to an in vivo hemodynamic flow rate.

4. The system of claim 2 wherein the continuous flow has a pressure that is substantially equivalent to an in vivo hemodynamic pressure.

5. The system of claim 2 wherein the continuous flow is adjustable to a predetermined setting.

6. The system of claim 2, further comprising a recirculating system configured for providing a recirculating flow of the continuous flow.

7. (canceled)

8. (canceled)

9. (canceled)

10. The system of claim 1 wherein the one or more micro-needles are fluidly
coupled.

11. The system of claim 1, further comprising:
a top cover;
a micro-needle portion comprising the one or more micro-needles; and
a base,
wherein the micro-needle portion is coupled between the top cover and the base and wherein the chamber is formed by the coupling of the top cover, micro-needle portion and the base.

12. The system of claim 11, further comprising a membrane portion configured for holding the tissue, the membrane portion positioned between the micro-needle portion and the base portion.

13. (canceled)

14. The system of claim 1 wherein the one or more micro-needles comprises a material selected from the group consisting of silicon, a biodegradable polymer and a combination thereof.

15.-127. (canceled)

128. A method of perfusing a tissue contained by a chamber, the method comprising injecting a fluid into a portion of the tissue, wherein the fluid is injected through a micro-needle.

129. The method of claim 128, further comprising flowing the fluid continuously through the tissue.

130. The method of claim 129, further comprising recirculating the fluid.

131. (canceled)

132. The method of claim 133 wherein the tissue is a liver tissue and wherein the fluid is injected into a sinusoid.

133. The method of claim 128 wherein the tissue is selected from the group consisting of liver, adrenal, bladder, brain, colon, eye, heart, kidney, liver, lung, ovary, pancreas, prostate, skin, small intestine, spleen, stomach, testis, thymus, tumor, and uterus tissue.

134. The method of claim 128 wherein the fluid is selected from the group consisting of an oxygenated fluid, a nutrient-containing fluid and a combination thereof.

135. (canceled)

136. The method of claim 128 wherein the fluid comprises a predetermined amount of a factor selected from the group consisting of a growth factor, a differentiation factor, a metabolite, and a hormone.

137. (canceled)

138. The method of claim 128 wherein the fluid is injected through a plurality of micro-needles.

139. The method of claim 138 wherein two or more of the plurality of micro-needles are fluidly coupled.

140. The method of claim 128 wherein the injecting comprises contacting a portion of the tissue with a micro-needle comprising a tip end positioned about an inlet port fluidly coupled to the chamber.

141. The method of claim 128, further comprising embedding the tissue between a polydimethylsiloxane (PDMS) membrane and a member.

142. The method of claim 128 wherein the member is a cover slip.

143. (canceled)

144. (canceled)

145. The method of claim 128, further comprising at least partially embedding the tissue in a polydimethylsiloxane (PDMS) membrane.

146. The method of claim 145 wherein the membrane comprises a polydimethylsiloxane (PDMS) membrane.

147. (canceled)

148. (canceled)

149. (canceled)

150. The method of claim 130 wherein the recirculating comprises receiving a tissue-exiting fluid about an outlet port fluidly coupled to the chamber.

151. The method of claim 128 wherein the tissue is a previously preserved tissue.

152. The method of claim 151 wherein the previously preserved tissue is a cryopreserved tissue.

153.-181. (canceled)

182. The method of claim 128, wherein said method is a method of analyzing an effect of a factor on a tissue contained by a chamber, wherein the fluid that is injected into a portion of the tissue comprises the factor; and wherein the method further comprises assaying to determine the effect of the factor.

183-188. (canceled)

189. The method of claim 182 wherein the factor is selected from the group consisting of a compound, an oxygen tension, a temperature, and a shear flow.

190. The method of claim 182 wherein the assaying comprises a member selected from the group consisting of microscopic analysis of the tissue, bio-imaging, histochemically staining the tissue, determining secretion of a biomolecule, determining metabolism of a biomolecule, determining an expression of a protein, determining an activation of a protein, determining an oxygen tension, determining a temperature, determining a shear flow and determining an intracellular level of a metabolite.

191.-198. (canceled)

199. The method of claim 190 wherein the metabolite is
urea or ammonia.

200. The method of claim 140 wherein the portion comprises a sinusoid.

201. The method of claim 190 wherein the protein is selected from the group consisting of liver albumin, beta galactosidase, and cytochrome P450.

202. The method of claim 182, further comprising transfecting the tissue with one or more nucleic acids.

203. The method of claim 182, further comprising infecting the tissue with one or more microbes.

204. The method of claim 203 wherein the one or more microbes is each independently selected from the group consisting of a bacteria, a virus, and a yeast.

205. The method of claim 202 wherein each of the one or more nucleic acids is a nucleic acid independently selected from the group consisting of albumin, beta galactosidase, cytochrome P450, glutathione-S-transferase, sulfotransferase, and N-acetyltransferase.

206. The method of claim 182 wherein the effect of the factor is selected from the group consisting of:
(a) adsorption of the factor or an analyte by at least one cell of the tissue;
(b) distribution of the factor or an analyte in at least one cell of the tissue;
(c) metabolism of the factor or an analyte by at least one cell of the tissue;
(d) permeability of the factor or an analyte to a cell membrane of at least one cell of the tissue;
(e) elimination or secretion of the factor or an analyte by at least one cell of the tissue; and
(f) toxicity of the factor or an analyte on at least one cell of the tissue.

207.-219. (canceled)

220. A method of claim 128, wherein said method is a method of growing said tissue contained by said chamber.

221. (canceled)

222. (canceled)

223. (canceled)

224. The method of claim 220, further comprising co-culturing the tissue with a stem cell or progenitor cell.

225. The method of claim 224, further comprising providing a differentiation signal to promote differentiation of the stem cell or progenitor cell.

226. The method of claim 220 wherein the tissue is from a previously preserved tissue.

227. The method of claim 226 wherein the previously preserved tissue is a cryopreserved tissue.

228.-237. (canceled)

238. A kit comprising one or more tissues configured for use in a tissue system comprising at least one chamber for containing the one or more tissues, one or more outlet ports fluidly coupled to the at least one chamber, one or more inlet ports fluidly coupled to the at least one chamber, and one or more micro-needles each comprising a tip end, the tip end being positioned about the one or more inlet ports and configured for injecting a fluid into one or more portions of the one or more tissues.

239. The kit of claim 238, wherein the one or more tissues are contained by said one or more chambers configured for use in said tissue system.

240. The method of claim 130, wherein said method is a method of culturing a tissue, and wherein the recirculating comprises receiving a tissue-exiting fluid about an outlet port fluidly coupled to the chamber.
